(19) United States
(12) Patent Application Publication
(10) Pub. No.: US 2021/0183487 A1
(43) Pub. Date: Jun. 17, 2021

(54) COGNITIVE PATIENT CARE EVENT RECONSTRUCTION

(71) Applicant: KONINKLIJKE PHILIPS N.V., Eindoven (NL)

(72) Inventors: Douglas Henrique Teodoro, Sao Paulo (BR); Lucas De Melo Oliveira, Wilmington, MA (US); Ricardo Guimaraes Heiss, Sao Paulo (BR)

(21) Appl. No.: 16/074,308

(22) PCT Filed: Feb. 1, 2017
(86) PCT No.: PCT/EP2017/052126

§ 371 Date: Jul. 31, 2018

Related U.S. Application Data
(60) Provisional application No. 62/290,083, filed on Feb. 2, 2016.

(51) Int. Cl. G16H 15/00; G16H 30/20; G16H 40/60; G16H 40/20

(52) U.S. Cl. CPC G16H 15/00; G16H 40/20; G16H 40/60; G16H 30/20

(57) ABSTRACT

A system includes a computing system (102) a processor (104) that performs the following: establish syntactic interoperability with a plurality of healthcare data sources (114); extract health care episode concepts from the sources, including concepts from a radiology report; classify the extracted concepts into cognitive classes, wherein the cognitive classes include: observation; evaluation; instruction and action; map the classified concepts to terminologies/ontologies; create a linked list of the events, including observations, evaluations, instructions and actions, to be contextualized; reconstruct the events from the linked list using time and location to order the events in a predetermined way; receive a query, including a unique identifier; for the events; construct, in response to the query, an output in electronical format that includes the events organized according to the cognitive classes and indexed by time from the reconstructed events; and transmit the constructed output via a network to a remote device.

### FIELD OF THE INVENTION

The following generally relates to visualizing current and past relevant patient information at a point of care.

### BACKGROUND OF THE INVENTION

To assess a patient problem or make an intervention decision, physicians combine and correlate large amounts of information, such as the patient's status, symptoms and treatments, stored in the patient health record over time. An electronic medical record (EMR) electronically stores the patient health record. While EMRs are good to store information, they are not well-suited for providing information to physicians at the point of care. With EMRs, physicians browse through several modules or systems to reconstruct the patient history, increasing time spent on non-care related tasks and reducing the room for effective patient care. Amongst others, the difficulties to access relevant and meaningful information contribute to the low impact of EMRs on the quality of care.

Due to the need for concise, meaningful and efficient ways to access and visualize patient's care information during encounters, the literature presents some systems that provide consolidated patient information varying over time. Knave II provides an interface, where many health care events can be visualized over time using a domain ontology browser. TimeLine provides a more elaborated interface, where all events of the treatment are captured over time in a single view and classified according to several care concepts, such as imaging, ischemia and cardiology. While these systems are able to consolidate and present information in a single, easily accessible place, in general they fail to provide this information in a meaningful way since they are unable to capture the cognitive communication processes of health professionals.

### SUMMARY OF THE INVENTION

Aspects of the present application address the above-referenced matters and others.

According to one aspect, a system includes a computing system with a memory device configured to store instructions, including a cognitive patient care event reconstruction module, and a processor that executes the instructions. The instructions cause the processor to: establish syntactic interoperability with a plurality of healthcare data sources; extract health care episode concepts from the plurality of healthcare data sources, including concepts from a radiology report; classify the extracted concepts into cognitive classes, wherein the cognitive classes include: observation; evaluation; instruction and action; map the classified concepts to terminologies/ontologies; create a linked list of the events, including observations, evaluations, instructions and actions, to be contextualized; reconstruct the health care episode events from the linked list using time and location to order the events in a predetermined way; receive a query, including a unique identifier; for the health care episode events; construct, in response to the query, an output in electronical format that includes the health care episode events organized according to the cognitive classes and indexed by time from the reconstructed events; and transmit the constructed output via a network to a remote device, resulting in the remote device visually presenting the constructed output in an interactive graphical user interface.

In another aspect, a method includes establishing, with a processor of a computing system, syntactic interoperability with a plurality of healthcare data sources; extracting, with the processor, health care episode events from the plurality of healthcare data sources; classifying, with the processor, the extracted concepts into cognitive classes; mapping, with the processor, the classified concepts to terminologies/ontologies; creating, with the processor, a linked list of the events to be contextualized; and reconstructing, with the processor, the health care episode events from the linked list using time and location to order the events in a predetermined way.

In another aspect, a non-transitory computer readable medium is encoded with computer executable instructions, which, when executed by a processor of a computer, cause the computer to: establish syntactic interoperability with a plurality of healthcare data sources; extract health care episode events from the plurality of healthcare data sources; classify the extracted health care episode events across observation; evaluation; instruction and action cognitive classes; map the classified health care episode events to terminologies/ontologies; create a linked list of the health care episode events to be contextualized; reconstruct the health care episode events from the linked list using time and location to order the events in a predetermined way; receive a query, including a unique identifier; for the health care episode events; construct, in response to the query, an output in electronical format that includes the health care episode events organized according to the cognitive classes and indexed by time from the reconstructed events; and transmit the constructed output via a network to a remote device, which causes the remote device to visually present the constructed output.

Still further aspects of the present invention will be appreciated to those of ordinary skill in the art upon reading and understand the following detailed description.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 schematically illustrates an example system including a computing system with a cognitive patient care event reconstruction module.

FIG. 2 schematically illustrates a non-limiting example of the cognitive patient care event reconstruction module in connection with data sources and a patient care navigation view.

FIG. 3 shows an example patient care navigation view that presents events and their relationships organized into cognitive knowledge classes.

FIG. 4 shows another example where the patient care navigator view presents events and their relationships organized into cognitive knowledge classes.

FIG. 5 shows an example of the patient care navigator view with mouse over and on click summary information.

FIG. 6 shows another example of the patient care navigator view with mouse over and on click summary information.

FIG. 7 shows another example of the patient care navigator view with mouse over and on click summary information.

FIG. 8 shows an example of the patient care navigator view for a selected observation.

FIG. 9 shows an example of the patient care navigator view for longitudinal observation.

FIG. 10 illustrates an example method in accordance with an embodiment herein.

### DETAILED DESCRIPTION OF EMBODIMENTS

The following describes a cognitive patient care event reconstruction approach that provides consolidated patient information visualization evolving over time with state-of-the-art techniques for clinical information modelling to improve EMR patient information data accessing and empower physicians during encounters.

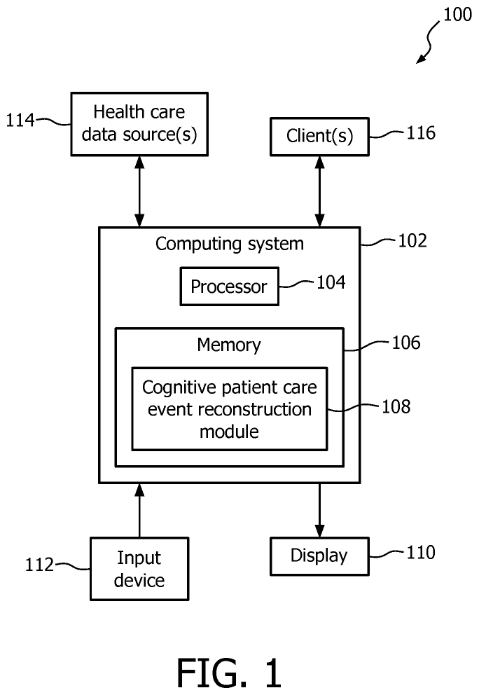

FIG. 1 illustrates a system 100. The system 100 includes a computing system 102 with at least one processor 104 (e.g., a microprocessor, a central processing unit, etc.) that executes at least one computer readable instruction stored in a computer readable storage medium (“memory”) 106, which excludes transitory medium and includes physical memory and/or other non-transitory medium. The instruction, in this example, includes a cognitive patient care event reconstruction module 108 with corresponding computer executable instructions. The computing system 102 also includes output device(s) 110, such as a display monitor, portable memory, a network interface, etc., and an input device(s) 112 such as a mouse, keyboard, a network interface, etc.

One or more healthcare data sources 114 provide data such as health care events to the computing system 102. A health care event, as utilized herein, is as any care event associated to a patient's episode of care and fall in one of the classes defined herein: observation, evaluation, instruction or action. For example, a health care event can be an observation of a lab exam, a patient diagnosis by the physician (evaluation), a medicine prescription (instruction), a microbiology test (action), etc. Examples of healthcare data sources 114 include imaging systems such as a positron emission tomography (PET), computed tomography (CT), single photon emission tomography (SPECT), magnetic resonance imaging (MRI), a combination thereof and/or other imaging scanner. Other examples include repositories such as a picture archiving and communication system (PACS), a radiology information system (RIS), a hospital information system (HIS), an electronic medical record (EMR), and/or other data repository. Other types of healthcare data sources 114 are also contemplated herein.

One or more clients 116 interact with the computing system 102. A client can be another computing device such as a computer, a laptop, a web-based application, a smartphone, a PACS, etc. A client 116 can communicate with the computing system 102 via a hard wire (e.g., a cable, etc.) and complementary electro-mechanical interfaces and/or wireless interfaces, using an application programming interfaces (API) and/or otherwise. As described in greater detail below, a client 116 queries the cognitive patient care event reconstruction module 108 for healthcare events of an individual (e.g., via a unique identification) and visually displays returned information via an interactive graphical user interface displayed via a display monitor.

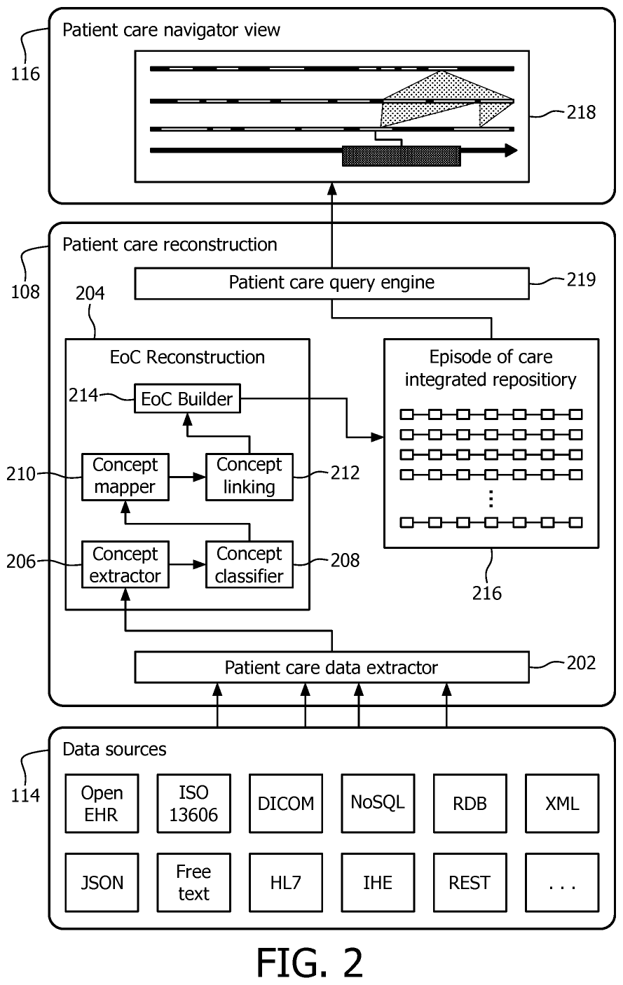

The instructions of the cognitive patient care event reconstruction module 108, when executed by the at least one processor 104, cause the at least one processor 104 to automatically capture and organize patient information following a cognitive clinical information model. This includes classifying healthcare events into cognitive classes: observation, evaluation, instruction, and/or action, and correlating the events so that patient care information can be easily accessed, contextualized and interpreted. Machine learning and natural language processing algorithms can be used to identify, classify and link care events. An example of the cognitive patient care event reconstruction module 108 is described in greater detail below in connection with FIG. 2. In one instance, the approach described herein overcomes the problem of accessing current and past relevant patient information at the point of care to foster physician decision making, and provides fast and meaningful access to patient data throughout the care process.

FIG. 2 schematically illustrates a non-limiting example of the cognitive patient care event reconstruction module 108 in connection with the data sources 114 and the client 116.

The cognitive patient care event reconstruction module 108 includes a patient care data extractor module 202. This module provides technical and syntactic interoperability to the healthcare data sources 114. In one instance, data from multiple data sources 114 are heterogeneous, with different data types, data models, formats and semantics. This module provides interfaces with the different data sources 114, homogenizing APIs and connection protocols, to extract events associated to the health care episode. It also converts the different data models into a single and flexible document model, based on standard syntax, such as JavaScript object notation (JSON), resource description framework (RDF), etc.

The cognitive patient care event reconstruction module 108 further includes an episode of care (EoC) reconstruction module 204 and an episode of care (EoC) integrated repository 216. This module includes several sub-modules that allow the episode of care events to be identified, classified into a cognitive model, mapped to standard terminologies or ontologies, and sequentially connected. In the illustrated embodiment, this module includes five sub-modules: a concept extractor sub-module 206, a concept classifier sub-module 208, a concept mapper sub-module 210, a concept linking sub-module 212, and an episode of care (EoC) builder sub-module 214.

The concept extractor sub-module 206 extracts healthcare data from the data sources 114 using the patient care data extractor module 202. For structured data attributes, this module simply calls the patient care data extractor module 202 for a given patient identifier. For unstructured data, such as commonly found in radiology and ultrasound reports, data is further processed using natural language processing (NLP) algorithms (e.g., stemming and lemmatization, part-of-speech tagging and chunking, phrase extraction and named entity recognition) to extract the concepts present in the text. For example, the following shows part of an example sample ultrasound report.

US ABDOMEN LTD SINGLE ORGAN

CLINICAL INFORMATION: 62-year-old male with cirrhosis.
COMPARISON: None
FINDINGS:
LIVER: Enlarged, measuring 19 cm in length.
Echotexture is slightly coarsened.
No focal masses identified.
BILIARY TRACT: Biliary tract is normal in caliber.
Gallbladder not visualized.
PANCREAS: Limited without gross abnormality.
SPLEEN: Enlarged at 14 cm without mass.
RIGHT KIDNEY: Both kidneys are echogenic.
OTHER: Extensive ascites.
IMPRESSION: Hepatosplenomegaly with extensive ascites.
Echogenic kidneys.

For the text passage “IMPRESSION: Hepatosplenomegaly with extensive ascites. Echogenic kidneys.” from this report, the concept extractor sub-modules 206 extracts the concepts “hepatosplenomegaly”, “ascites” and “echogenic kidneys”.

The concept classifier sub-module 208 classifies the concepts extracted by the concept extractor sub-modules 206 into the cognitive classes of the healthcare process: 1) observation, 2) evaluation, 3) instruction and 4) action. For example, for the above ultrasound report with the structure:

US . . .

CLINICAL INFORMATION: . . .

COMPARISON: . . .

FINDINGS: . . .

IMPRESSION: . . .

the concept classifier sub-module 208 automatically identifies the heading or parts of reports (or argumentative moves) from where the concepts were extracted and classifies them according to the cognitive classes. In this example, the ultrasound exam heading (“US ABDOMEN . . . ”) would be classified as the action performed, the “FINDINGS” heading would be the observations from the action, and the concepts extracted from the “IMPRESSION” heading would be classified as evaluation. If the data are from a structured database, this task is simplified. For example, the attributes could be manually mapped to the different cognitive classes. For instance, all the concepts coming from the “primary diagnosis” column of an “episode of care” table would be classified as “evaluation”.

The concept mapper sub-module 210 maps the concepts classified by the concept classifier sub-module 208 to standard terminologies/ontologies. For example, the concept “ascites” would be mapped to K70.31 in (International Classification of Diseases) ICD-10. The ICD is the international standard diagnostic tool for epidemiology, health management and clinical purposes. This sub-module can be implemented using string distance (e.g., Levenshtein) and/or concept expansion and machine learning (e.g., support vector machine (SVM) and Neural Network). This allows concepts to be semantically standardized, so that they can be concisely displayed in interfaces.

Other terminologies/ontologies include SNOMED Clinical Terms (CT), Logical Observation Identifiers Names and Codes (LOINC), and RxNorm. SNOMED CT is a systematically organized computer processable collection of medical terms providing codes, terms, synonyms and definitions used in clinical LOINC is a database and universal standard for identifying medical laboratory observations. RxNorm is a name of a US-specific terminology in medicine that contains all medications available on US market and can be used in personal health records applications. Other terminologies/ontologies are also contemplated herein.

The concept linking sub-module 212 creates a linked (or associated) list of events (observations, evaluations, instructions and actions) so that patient care information can be contextualized. This is implemented using a relational structure of the datasets or time dependencies between events when no clear link is available in the data. For example, in the ultrasound report above, the structure of the information can be used to create the associations, where it is easy to infer that the ultrasound (action) led to a finding (observation) that led to an impression (evaluation). However, it might be that this information is not readily connected in the data sources. For example, a physician can prescribe an antibiotic before having the result of the microbiology test. In these cases, time between events could be used to connect them. A posterior observation of an abnormal amount of bacteria in the microbiology test can lead to a bacterial infection diagnosis, which had originally led to the antimicrobial treatment action. A difference of few days between the beginning of the antibiotic treatment and the result of the microbiology test could be used to connect these events.

The episode of care (EoC) builder sub-module 214 gathers the different events belonging to a patient's episode of care and constructs an array structure that stores all this information in the episode of care (EoC) repository 216. The episode of care (EoC) builder sub-module 214 reconstructs the episode of care information using time and location features to order the episode of care events in a meaningful way. It provides a connector to the episode of care (EoC) repository 216, allowing data to be loaded into it. Data streams are routinely loaded into the central repository using time stamps of the source datasets.

The episode of care (EoC) repository 216 stores all information related to a patient's episode of care found within the healthcare institution (and eventually outside, e.g., public healthcare data). This repository aggregates data from several healthcare data sources 114 to create a unified register with the patient population flow, encoded in the episodes of care. In this context, an episode of care encodes all healthcare data relevant to the patient care, including i) patient demographics, such as age range and gender, ii) clinical events, such as procedures, diagnoses, lab exams and medications, and iii) administrative and operational information, such as the locations the patient stayed in the institution, the respective time, and the physicians that treated the patient. To capture the document model of the episode of care, which is largely derived from the patient health record document, this repository could be backed by a NoSQL database, providing high model flexibility and retrieval performance.

The cognitive patient care event reconstruction module 108 further includes a patient care query engine 219. This module provides means to actually access the patient data so that it can be displayed in a user interface. The module receives a patient identifier and optionally a period, and outputs all the data stored for the patient, organized according to the cognitive information classes, and indexed by time.

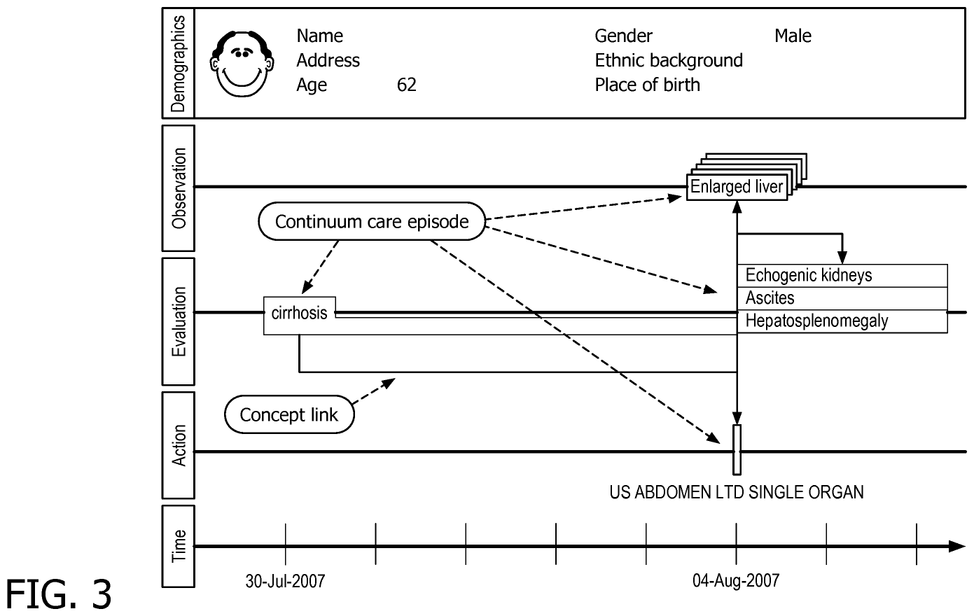

The client 116, in this example, includes a patient care navigator view 218. The patient care navigator view 218 interface is where the patient information is accessed and visualized by the physician. This view uses the patient care query engine 219 to extract information about a single patient and organizes the display taking into account the healthcare cognitive information flow, i.e., observation, evaluation, instruction and action. FIG. 3 shows an example of how this interface could be implemented to represent the patient's healthcare information from the sample ultrasound report discussed above. In this example, the instruction dimension is not represented and, in this case, the action dimension can be taken as surrogate for the instructions events. This interface could be implemented, e.g., using HTML5 technologies, a visualization library written in Java Script, etc.

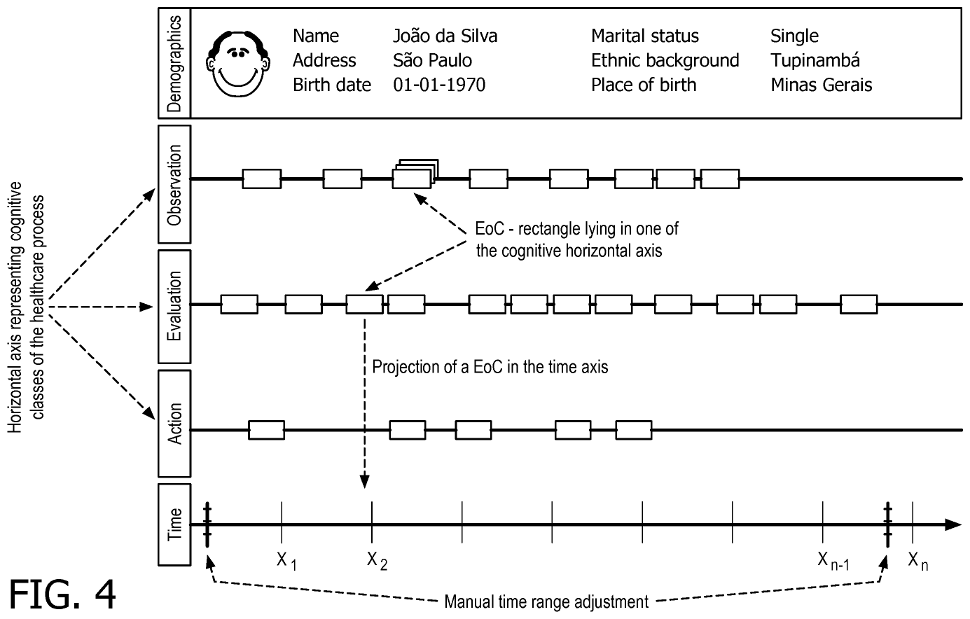

FIGS. 4-9 illustrate other examples of the patient care navigator view 218.

With reference to FIG. 4, the patient care navigator view 218 presents automatically to the user (e.g., a physician) the events within the patent event of care reconstruction module 204 of a patient, and their relationships, organized into cognitive knowledge classes according to the model. In one instance, the events were mapped and organized in five different axes. The first four horizontal axes represent the demographic information and three different events of the healthcare process (observation, evaluation, and action). The fourth axis, time bar, represents the time when an event occurred. Each event displayed in the patient care navigator view 218 is represented by a rectangle lying in their respective cognitive horizontal axis. The projection of each rectangle in the time bar indicates the time in which that event happened.

The level of details of the information displayed in the patient care navigator view 218 can be defined by the dynamic selection of the time range in the time bar. By narrow down or expand the range dates in the time bar, the user can reduce/increase the amount of information displayed (size of the rectangles, amount of information show in the rectangle and weight of the link between rectangle. For X-ray image observation event for example, the incremental amount of information displayed in the rectangle can vary from a single icon in to the complete reason for exam and study protocol (e.g., “XR PORT CHEST 1V—20 old female with sickle cell and sudden onset of left-sided weakness and paraesthesia”). This gives the user the option to see a complete overview of the patient history or only a small date range. The range is delimited by two dates (initial and final) indicated in the time bar.

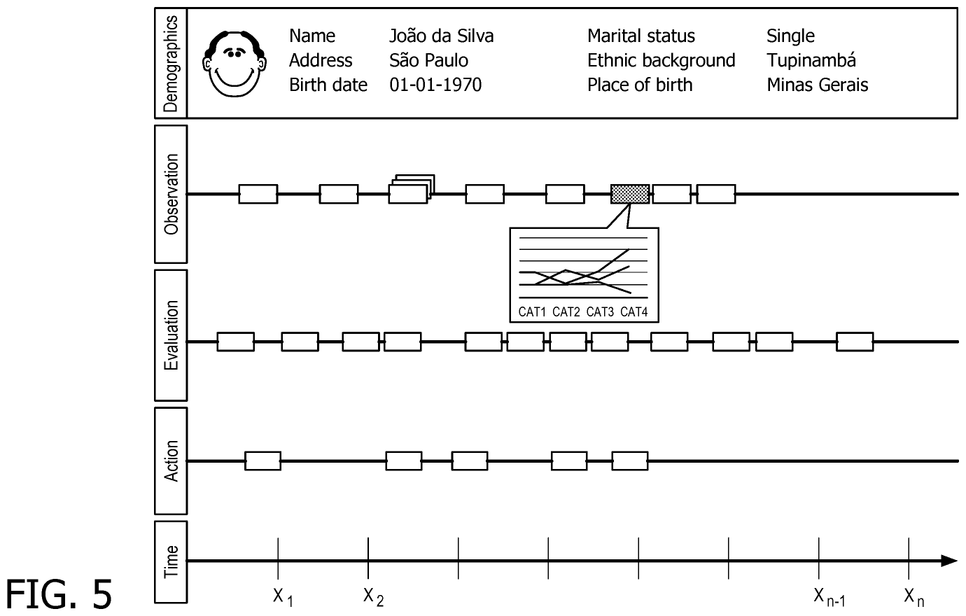

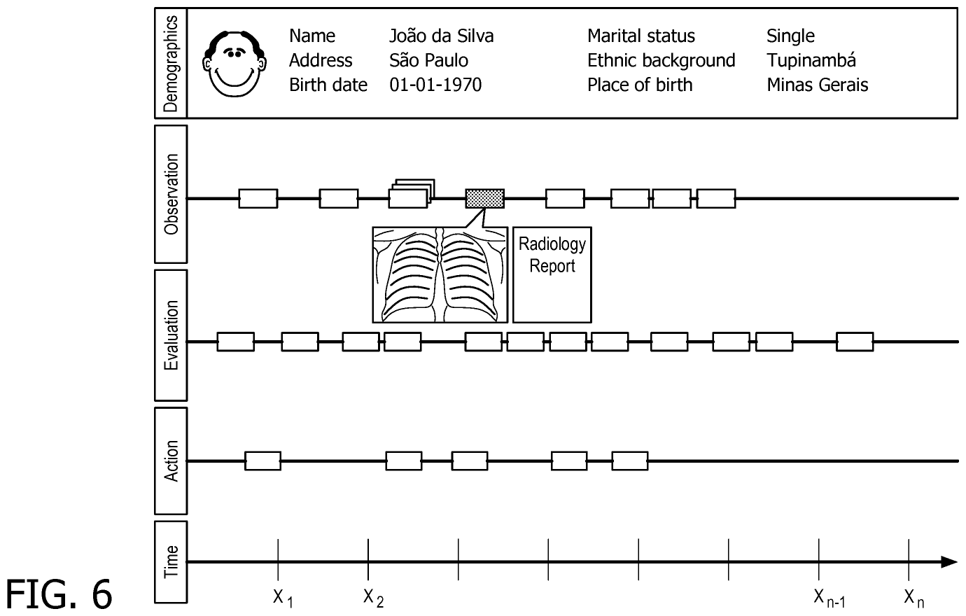

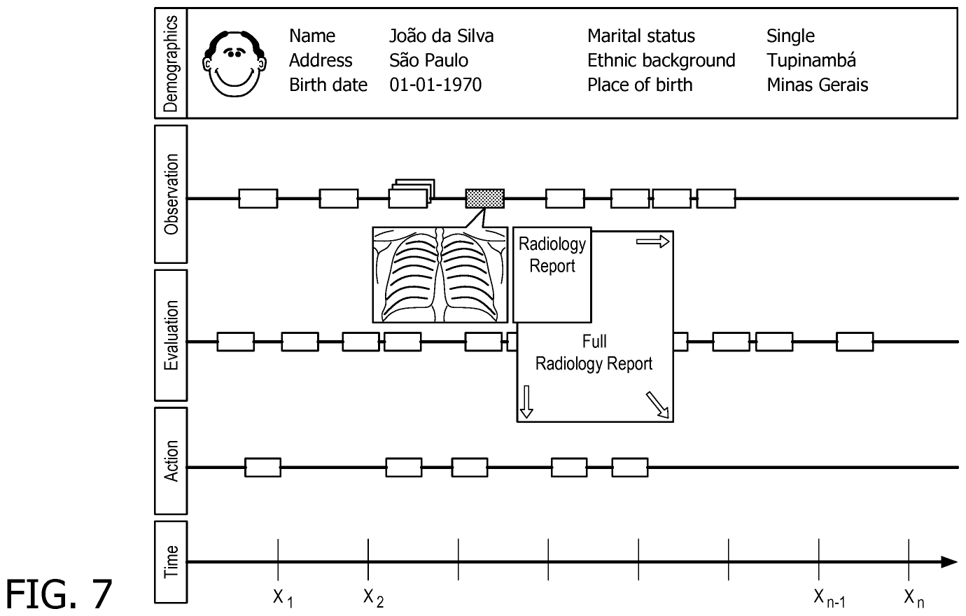

FIGS. 5, 6 and 7 show examples of the patient care navigator view 218 with mouse over and on click summary information. By mouse hovering a care event the user can have a preview of the information, e.g., text, graph or image (FIG. 5). In one instance, if the event stored represents a chest CT image exam, the user can see a snapshot of the radiology report (FIG. 6). By clicking in such event, the user can have a more detailed view of that event and interact with the displayed information (FIG. 7). Considering the same CT example describe above, the user can expand the radiology report summary to getting more detail of the information stored in that event.

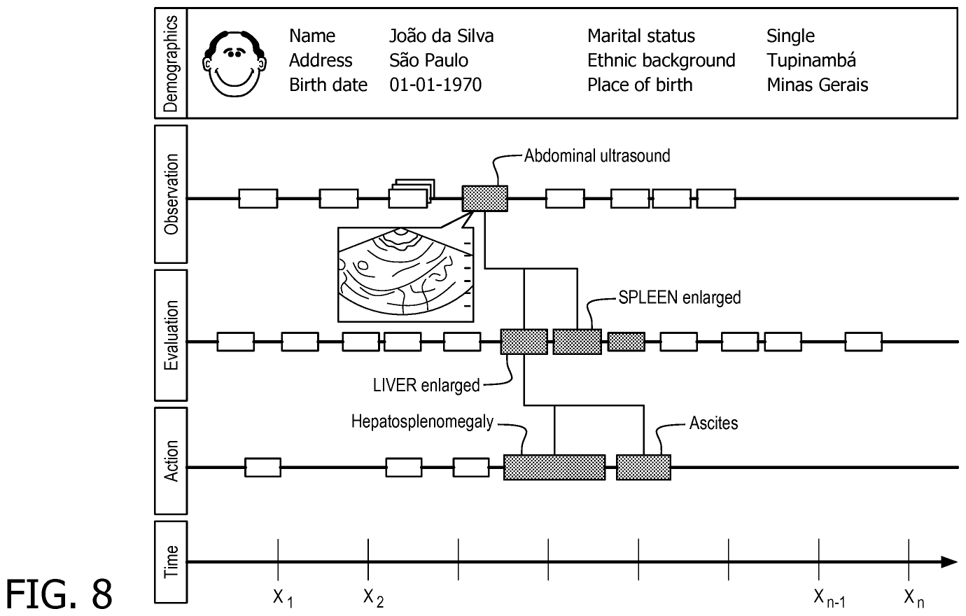

FIG. 8 shows an example of the patient care navigator view 218 for a selected observation. This visualization displays the evaluation(s) and/or action(s) associated with a selected observation. For example, if the radiology report for an abdominal ultrasound imaging study (i.e., the observation) mentioned that the “liver” and the “spleen” are enlarged (i.e., the evaluations), gave a diagnosis of “hepatosplenomegaly” and “ascites” (i.e., the actions) due to the enlarged liver, the patient care navigator view 218 would automatically highlight the rectangles associated with “liver enlarged”, “spleen enlarged”, “hepatosplenomegaly” and “ascites” and the existing link between them.

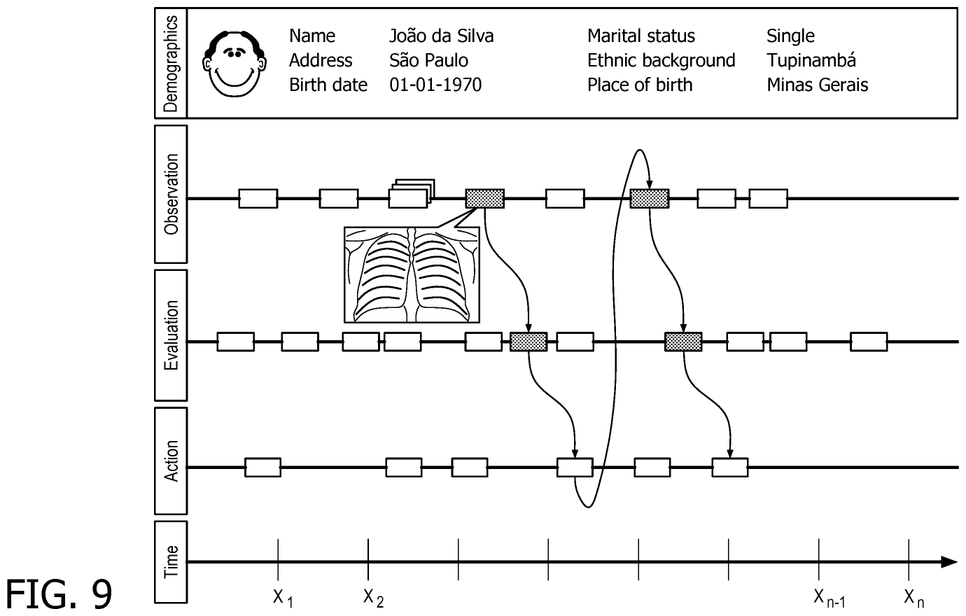

FIG. 9 shows an example of the patient care navigator view 218 for longitudinal observation. This visualization provides a longitudinal view of an observation event. In some cases, a clinical finding mapped by an evaluation (e.g., a pulmonary nodule or a peritonitis) can be evaluated several times to verify the severity or progression of this clinical finding. This created a close loop between observation evaluation action observation. For example, after a pulmonary nodule (evaluation) is noted in an image study (observation), the radiologist can schedule a series of follow-up imaging exam (action) to track progression of the nodule or get more details of the nodule. In the follow-up imaging exam (e.g., CT or MRI), the radiologist can write a report giving more details about the pulmonary nodule previously noted and request annual additional exams for the next five years to follow the progression of that nodule. In this case, the longitudinal observation can show all the sequence of exam in a fashion manner by highlighting the path of the pulmonary nodule in the patient care navigator view 218.

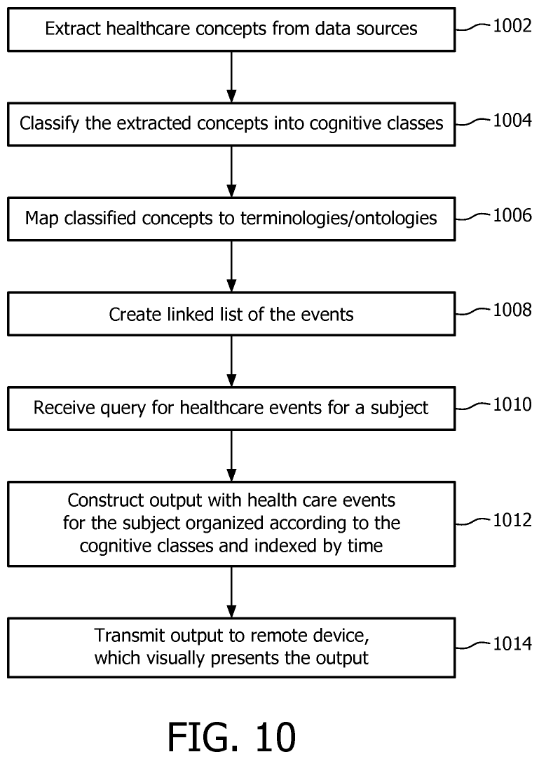

FIG. 10 illustrates an example method in accordance with an embodiment herein.

It is to be appreciated that the ordering of the acts in the methods described herein is not limiting. As such, other orderings are contemplated herein. In addition, one or more acts may be omitted and/or one or more additional acts may be included.

At 1002, healthcare data concepts are extracted from healthcare data sources, as described herein and/or otherwise.

At 1004, the extracted healthcare concepts are classified into a predetermined set of cognitive classes, as described herein and/or otherwise.

At 1006, the classified concepts are mapped to terminologies and/or ontologies, as described herein and/or otherwise.

At 1008, a linked list of the events is created to contextualize the patient care information, as described herein and/or otherwise.

At 1010, a query for healthcare events of a single patient is retrieved, as described herein and/or otherwise.

At 1012, an output is constructed and includes the health care events for the subject organized according to the cognitive classes and indexed by time from the reconstructed events, as described herein and/or otherwise.

At 1014, the constructed output is transmitted to a remote device, which causes the remote device to visually present the constructed output, as described herein and/or otherwise.

The method herein may be implemented by way of computer readable instructions, encoded or embedded on computer readable storage medium, which, when executed by a computer processor(s), cause the processor(s) to carry out the described acts. Additionally or alternatively, at least one of the computer readable instructions is carried by a signal, carrier wave or other transitory medium.

In one instance, any of the plurality of data sources 114 causes the cognitive patient care event reconstruction module 108 to retrieve and/or receive healthcare data from any of the plurality of data sources 114. For example, where the plurality of healthcare data sources 114 includes an imaging system, the imaging system can transmit a signal to the cognitive patient care event reconstruction module 108 indicating the new image data is available. In response thereto, the cognitive patient care event reconstruction module 108 is invoked to extract healthcare data as described herein. In one instance, the signal controls the cognitive patient care event reconstruction module 108 to extract the data.

In another instance, the cognitive patient care event reconstruction module 108 causes the client 116 to retrieve and/or receive constructed output (e.g., the health care episode events organized according to the cognitive classes and indexed by time) in electronical format and display or visually present it. For example, where the cognitive patient care event reconstruction module 108 receives, modifies, etc. data, the cognitive patient care event reconstruction module 108 transmits a signal the client 116 indicating this. In response thereto, the cognitive patient care event reconstruction module 108 pushes the constructed output to the client device 116 or the client device 116 pulls the constructed output, and this causes the client device 116 to visually present the constructed output.

The approach described herein can improve computing system performance. For instance, it can reduce the number of processing cycles required to construct a meaningful output. Furthermore, it efficiently stores the classified and linked in memory. In one instance, this enables fast and meaningful access to patient data, relative to a configuration where the cognitive patient care event reconstruction module 108 is omitted.

The invention has been described herein with reference to the various embodiments. Modifications and alterations may occur to others upon reading the description herein. It is intended that the invention be construed as including all such modifications and alterations insofar as they come within the scope of the appended claims or the equivalents thereof.

1. A system, comprising:
a computing system, including:
a memory device configured to store instructions, including a cognitive patient care event reconstruction module; and
a processor that executes the instructions, which causes the processor to:
establish syntactic interoperability with a plurality of healthcare data sources;
extract health care episode concepts from the plurality of healthcare data sources, including concepts from a radiology report;
classify the extracted concepts into cognitive classes, wherein the cognitive classes include: observation; evaluation; instruction and action;
create a linked list of the events, including observations, evaluations, instructions and actions;
reconstruct the health care episode events from the linked list using time and location to order the events in a predetermined way;
receive a query, including a unique identifier; for the health care episode events;
construct, in response to the query, an output in electronical format that includes the health care episode events organized according to the cognitive classes and indexed by time from the reconstructed events; and
transmit the constructed output via a network to a remote device, resulting in the remote device visually presenting the constructed output in an interactive graphical user interface.

2. The system of claim 1, wherein the plurality of healthcare data sources includes an imaging system, and the processor, in response to receiving a signal from the imaging system, where the signal indicates new image data is available, automatically extracts an imaging health care episode event from the imaging system.

3. The system of claim 1, wherein the remote device is a client, and the transmission of the constructed output to the client controls the client to visually present the constructed output.

4. The system of claim 1, wherein the processor establishes syntactic interoperability by providing interfaces with the plurality of healthcare data sources that homogenize application programming interfaces and connection protocols.

5. (canceled)

6. The system of claim 1, to wherein the processor extracts health care episode events from unstructured data attributes using a natural language processing algorithm to extract concepts in the text.

7. The system of claim 1, wherein the processor classifies the extracted concepts by automatically identifying section headings of the report from where the concepts were extracted and classifying the concepts based on the identified section heading.

8. The system of claim 7, wherein the processor classifies extracted concepts for an exam type heading as an action, classifies extracted concepts for a findings heading as an observation, and classifies extracted concepts for an impression exam heading as an evaluation.

9. (canceled)

10. The system of claim 1, wherein the processor links the list of events using a relational structure of datasets.

11. The system of claim 10, wherein the processor links an action, an observation and an evaluation, wherein the action led to the finding which caused the evaluation.

12. (canceled)

13. (canceled)

14. The system of claim 1, wherein the interactive graphical user interface presents the constructed output visually showing relationships between the events.

15. A method, comprising:
establishing, with a processor of a computing system, syntactic interoperability with a plurality of healthcare data sources;
extracting, with the processor, health care episode concepts from the plurality of healthcare data sources, including concepts from a radiology report;
classifying, with the processor, the extracted concepts into cognitive classes, wherein the cognitive classes include: observation; evaluation; instruction and action;
creating, with the processor, a linked list of the events, including observations, evaluations, instructions and actions;
reconstructing, with the processor, the health care episode events from the linked list using time and location to order the events in a predetermined way;
receiving, with the processor, a query, including a unique identifier, for the health care episode events;
constructing, in response to the query, an output in electronical format that includes the health care episode events organized according to the cognitive classes and indexed by time from the reconstructed events; and
transmitting the constructed output via a network to a remote device, resulting in the remote device visually presenting the constructed output in an interactive graphical user interface.

16. The method of claim 15, wherein the cognitive classes include: an observation class; an evaluation class; an instruction class and an action class.

17. The method of claim 15, further comprising:
receiving, with the processor, a query, including a unique identifier; for the health care episode events; and
constructing, with the processor and in response to the query, an output in electronical format that includes the health care episode events organized according to the cognitive classes and indexed by time from the reconstructed events.

18. The method of claim 17, further comprising:
transmitting, with the processor, the constructed output via a network to a remote device.

19. (canceled)

20. A non-transitory computer readable medium encoded with computer executable instructions, which, when executed by a processor of a computer, cause the computer to:
establish syntactic interoperability with a plurality of healthcare data sources;
extract health care episode concepts from the plurality of healthcare data sources;
classify the extracted health care episode events across observation; evaluation; instruction and action cognitive classes;
create a linked list of the health care episode events;
reconstruct the health care episode events from the linked list using time and location to order the events in a predetermined way;
receive a query, including a unique identifier; for the health care episode events;
construct, in response to the query, an output in electronical format that includes the health care episode events organized according to the cognitive classes and indexed by time from the reconstructed events; and
transmit the constructed output via a network to a remote device, which causes the remote device to visually present the constructed output.
